(19) United States
(12) Patent Application Publication
(10) Pub. No.: US 2008/0086166 A1
(43) Pub. Date: Apr. 10, 2008

(54) Minimally Invasive Surgical Assembly with Balloon Instrument

(76) Inventor: Sundaram Ravikumar, Briarcliff Manor, NY (US)

Correspondence Address: GORDON & JACOBSON, P.C., 60 LONG RIDGE ROAD, SUITE 407, STAMFORD, CT, 06902, US

(21) Appl. No.: 11/685,586

(22) Filed: Mar. 13, 2007

Related U.S. Application Data
(60) Provisional application No. 60/828,916, filed on Oct. 10, 2006.

(51) Int. Cl. A61M 25/10

(52) U.S. Cl. 606/192

(57) ABSTRACT

A minimally invasive surgical assembly broadly includes an outer hollow needle which has an outer diameter of at most 3.0 mm, and a coaxial surgical instrument having a shaft which extends through the outer hollow needle. The coaxial surgical instrument includes a balloon or inflatable bladder at the end of the shaft which may be inflated and deflated. The assembly preferably includes a first fixing element which is used to fix the relative location of the surgical balloon instrument and the needle. The assembly also preferably includes a second fixing element which moves relative to the needle and is located on the outside thereof and which is used to fix the relative location of the needle to the patient.

This application claims priority from U.S. provisional application No. 60/828,916 filed Oct. 10, 2006, which is hereby incorporated by reference in their entireties. This application is also related to U.S. Ser. No. 11/420,927 filed May 30, 2006, and U.S. Ser. No. 11/685,522 filed on an even date herewith, which are also hereby incorporated by reference in their entireties.

### BACKGROUND OF THE INVENTION

1. Field of the Invention

This invention relates broadly to surgical instruments and methods of their use. More particularly, this invention relates minimally invasive surgical assemblies incorporating a needle and a working device which extends through and beyond the needle and which can be retracted into the needle. The working device includes a balloon. The invention has particular application to laparoscopic-type surgery, although it is not limited thereto.

2. State of the Art

Over the last two decades, minimally invasive surgery has become the standard for many types of surgeries which were previously accomplished through open surgery. Minimally invasive surgery generally involves introducing an optical element (e.g., laparoscope or endoscope) through a surgical or natural port in the body, advancing one or more surgical instruments through additional ports or through the endoscope, conducting the surgery with the surgical instruments, and withdrawing the instruments and scope from the body. In laparoscopic surgery (broadly defined herein to be any surgery where a port is made via a surgical incision, including but not limited to abdominal laparoscopy, arthroscopy, spinal laparoscopy, etc.), a port for a scope is typically made using a surgical trocar assembly. The trocar assembly often includes a port, a sharp pointed element (trocar) extending through and beyond the distal end of the port, and at least in the case of abdominal laparoscopy, a valve on the proximal portion of the port. Typically, a small incision is made in the skin at a desired location in the patient. The trocar assembly, with the trocar extending out of the port is then forced through the incision, thereby widening the incision and permitting the port to extend through the incision, past any facie, and into the body (cavity). The trocar is then withdrawn, leaving the port in place. In certain circumstances, an insufflation element may be attached to the trocar port in order to insufflate the surgical site. An optical element may then be introduced through the trocar port. Additional ports are then typically made so that additional laparoscopic instruments may be introduced into the body.

Trocar assemblies are manufactured in different sizes. Typical trocar port sizes include 5 mm, 10 mm and 12 mm (available from companies such as Taut and U.S. Surgical), which are sized to permit variously sized laparoscopic instruments to be introduced therethrough including, e.g., graspers, dissectors, staplers, scissors, suction/irrigators, clamps, forceps, biopsy forceps, etc. While 5 mm trocar ports are relatively small, in some circumstances where internal working space is limited (e.g., children), it is difficult to place multiple 5 mm ports in the limited area. In addition, 5 mm trocar ports tend to limit movements of instruments inside the abdominal cavity to a great extent.

Further, while laparoscopic surgery has reduced the trauma associated with various surgical procedures and has concomitantly reduced recovery time from these surgeries, there always remains a desire in the art to further reduce the trauma to the patient.

One area of trauma associated with laparoscopic surgery identified by the inventor hereof as being susceptible of reduction are the scars which result from the trocar ports used. In many laparoscopic surgeries, three or more trocar incisions are made. For example, in laparoscopic hernia repair surgery, four trocar incisions are typically made, with one incision for insufflating the abdomen and inserting the optical device, two incisions for trocar ports for inserting graspers therethrough, and a fourth port for passing a stapler therethrough. Those skilled in the art and those who have undergone surgical procedures recognize that even the 5 mm trocar ports leave holes which must be stitched and which result in scars.

A second area of trauma associated with laparoscopic surgery identified by the inventor hereof as being susceptible of reduction relates to trauma resulting from the manipulation (angling) of the trocar ports required in order to conduct the surgery due to inexact placement. Angling of the port can cause tearing at the incision periphery.

Those skilled in the art will also appreciate that because of the number of trocar assemblies and laparoscopic tools used in laparoscopic surgery (most of which are disposable because of the cost and complications associated with autoclaving), the cost of laparoscopic surgery is high. Thus, there always remains a desire in the art to provide lower cost laparoscopic tools.

### SUMMARY OF THE INVENTION

It is therefore an object of the invention to provide a minimally invasive surgical assembly which reduces trauma to the patient relative to presently used systems.

It is another object of the invention to provide a minimally invasive surgical assembly which is simple and inexpensive relative to presently used systems.

It is a further object of the invention to provide a minimally invasive surgical assembly which utilizes a 3 mm or smaller incision/port device and a surgical balloon instrument.

It is also an object of the invention to provide a minimally invasive surgical assembly which will not scar a patient.

It is an additional object of the invention to provide a minimally invasive surgical assembly utilizing effective surgical balloon instruments which are inserted into a 3 mm or smaller port device.

It is still another object of the invention to provide a minimally invasive surgical assembly with reduced number of parts.

In accord with these objects, which will be discussed in detail below, a minimally invasive surgical assembly according to the invention broadly includes an outer hollow needle which has an outer diameter of substantially 2.5 mm or smaller (the term “substantially”, for purposes of this application meaning ±20%), and a coaxial surgical instrument having a shaft which extends through the outer hollow needle. The coaxial surgical instrument includes a balloon or inflatable bladder at the end of the shaft which may be inflated and deflated. The assembly preferably includes a first fixing element which is used to fix the relative location of the surgical balloon instrument and the needle. The assembly also preferably includes a second fixing element which moves relative to the needle and is located on the outside thereof and which is used to fix the relative location of the needle to the patient. The second fixing assembly may include an anchoring element which permits the needle to be held at different angles relative to the patient.

According to one embodiment of the invention, the surgical balloon instrument and needle are sized so that at least a portion of the shaft of the balloon instrument interferingly slides against the inner surface of the needle, thereby forming a seal which is effective against desufflation.

The surgical assembly of the invention may be used during laparoscopic surgery instead of using an extra trocar and laparoscopic instrument. In particular, with the surgical balloon instrument partially inserted in the needle (i.e., with the balloon at least partially withdrawn inside the needle) and optionally locked relative to each other by the first fixing element, the needle is used to puncture the skin and advance into the body (e.g., the abdomen). At a desired location (typically under guidance of an already inserted scope), the movement of the needle is stopped. The surgical instrument is then unlocked (if previously locked) and advanced until the balloon is located beyond the tip of the needle. The needle and surgical instrument may then further advanced to a desired location. When desired, the balloon can be inflated without concern that it will be punctured by the needle. The first fixing element may then be used to fix the needle relative to the balloon to prevent inadvertent puncture. If desired, the needle with the surgical balloon instrument fixed relative thereto may be manipulated relative to the body wall (e.g., to lift, push, pull, dissect, or otherwise move a structure). When the surgical assembly is in a desired location in the body, the second fixing element is slid along the needle and into engagement with the skin of the patient, thereby fixing the surgical instrument at a desired location in the body. At any time, when it is desired to remove the surgical assembly from the body (e.g., when surgery is completed), the balloon may be deflated. The surgical assembly can be pulled out of the body (preferably with the surgical balloon instrument first moved backward relative to the needle to retract and locate the balloon inside the needle) leaving just a small puncture mark which will often heal without a scar.

The surgical assembly of the invention thereby accomplishes the objects of the invention with a minimum number of parts and may be used to replace expensive trocar assemblies and laparoscopic instruments.

Additional objects and advantages of the invention will become apparent to those skilled in the art upon reference to the detailed description taken in conjunction with the provided figures.

### BRIEF DESCRIPTION OF THE DRAWINGS

FIG. 1 is a perspective view of the minimally invasive surgical assembly of the invention in conjunction with a syringe, tubing and a valve and with the balloon instrument shown inflated in a first extended position relative to the needle.

FIG. 2 is a schematic diagram of the minimally invasive surgical assembly with the balloon instrument shown deflated in the first extended position relative to the needle.

FIG. 3 is a schematic diagram of the assembly of FIG. 2 with the balloon instrument shown deflated in a second withdrawn position relative to the needle.

FIG. 4 is a schematic diagram of the distal end of a balloon instrument utilizing a first type of balloon.

FIG. 5A is a schematic diagram of the distal end of a balloon instrument utilizing a second type of balloon shown in an uninflated position.

FIG. 5B is a schematic diagram of the balloon instrument of FIG. 5A with the balloon shown in an inflated position.

### DETAILED DESCRIPTION OF THE PREFERRED EMBODIMENTS

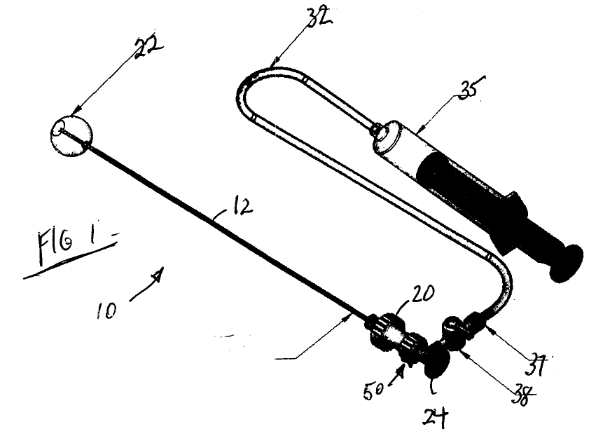

A minimally invasive surgical assembly 10 according to the invention and as seen in FIGS. 1-3 broadly includes an outer hollow needle 12 which has an outer diameter of substantially 2.5 mm (0.1 inches) or smaller, and a coaxial surgical instrument 14 having a shaft 15 which extends through the outer hollow needle. The needle 12 has a sharpened distal end 18 which is angled at about 35° relative to a longitudinal axis of the needle, and a proximal end having a knob or handle 20 for holding and manipulation of the needle. The inside diameter of the needle is approximately 2.0 mm (0.08 inches) and the wall thickness of the needle is approximately 0.25 mm (0.01 inch). The needle is typically between 10 and 30 cm long, and more typically between 13 and 18 cm long (although other sizes could be used, depending upon the surgery involved, and typically larger for obese patients and smaller for infants and small children), and is preferably made from stainless steel, although other materials could be utilized.

The coaxial surgical instrument 14 shown in FIG. 1 is an inflatable balloon instrument and includes the shaft 15 which is hollow, an inflatable balloon or bladder 22 at the distal end of the hollow shaft 15, a handle or knob 24 at the proximal end of the shaft, and means 26 at the proximal end of the shaft for coupling to a fluid source. As shown in FIG. 1, the means for coupling to a fluid source is a luer slip or fitting 27 which can receive a tube 32 of desired length (e.g., eighteen inches) which is coupled to a syringe 35, or which can alternatively be accessed by the syringe itself. Also shown in FIG. 1 is an optional valve 38 which is located between the luer 27 and the hollow shaft 15. In this manner, with the valve 38 in an open position, the syringe 35 can be used to inflate the balloon 22. The valve 38 may then be closed, and the balloon 22 will remain inflated even if the syringe 35 is disconnected from the assembly. Deflation of the balloon 22 may then be accomplished by opening the valve 38. The shaft 15 of the balloon instrument 14 must be long enough to permit the balloon or bladder 22 of the balloon instrument to extend out of the needle as seen in FIG. 1. The shaft of the balloon instrument 14 is preferably made from stainless steel, although other materials could be utilized for all or part of the instrument 14.

According to one aspect of the preferred embodiment of the invention, the balloon instrument 14 and needle 12 are sized so that at least a portion of the shaft 15 of the balloon instrument 14 snugly slides against the inner surface of the needle 12, thereby forming a seal which is effective against desufflation. Thus, at least a portion of the outer diameter of the shaft 15 proximal of the balloon 22 is approximately 1.99 mm, or about 0.01 mm smaller than the inner diameter of the needle. This small difference in diameters results in a sliding snug fit which can be felt as a drag and which effectively acts as a seal against desufflation (of the abdominal cavity). Alternatively, the needle may include an internal gasket or seal or grease which seals against the outer diameter of the shaft.

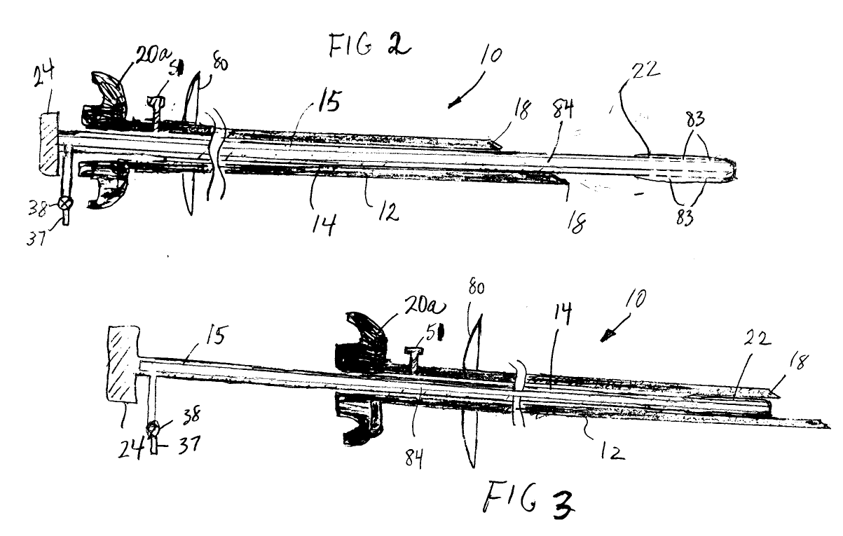

As indicated in FIG. 1, according to the preferred embodiment, the assembly 10 of the invention includes a first fixing mechanism, element, or system 50 which is used to fix the relative location of the balloon instrument 14 and the needle 12. As disclosed in the previously incorporated U.S. Ser. No. 11/420,927, the fixing system 50 may take any of many different embodiments. Thus, a screw 51 (as shown in FIGS. 2 and 3) which extends through a threaded radial hole in the needle 12 or its handle may be used to fix the balloon instrument 14 relative to the needle 12. If desired notches (not shown) may be provided on the shaft 15 of the balloon instrument 14 to receive the end of the screw. Alternatively, as described in previously incorporated U.S. Ser. No. 11/420,927, a cam element (not shown) can be used which is rotatingly coupled to the needle handle by a pin (not shown). When in a first orientation, the cam element permits a rear portion of the shaft 15 of the balloon instrument 14 to move in an uninhibited manner. When in a second orientation, the cam element engages the rear portion of the shaft 15 and holds it fixed relative to the needle handle and needle 12. Alternatively, any of the other fixing systems disclosed in previously incorporated U.S. Ser. No. 11/420,927 or U.S. Ser. No. 11/685,522 may be used, or any other fixing system suitable for fixing the relative position of the balloon instrument 14 relative to the needle.

The assembly may also be provided with a second fixing element 80 (FIGS. 2 and 3) which moves relative to the needle and is located on the outside thereof and which is used to fix the relative location of the needle to the patient. Any of various second fixing elements may be provided including those disclosed in previously incorporated U.S. Ser. No. 11/420,927, those disclosed in previously incorporated U.S. Ser. No. 11/685,522, as well as other fixing elements which will serve to fix the relative location of the needle to the patient.

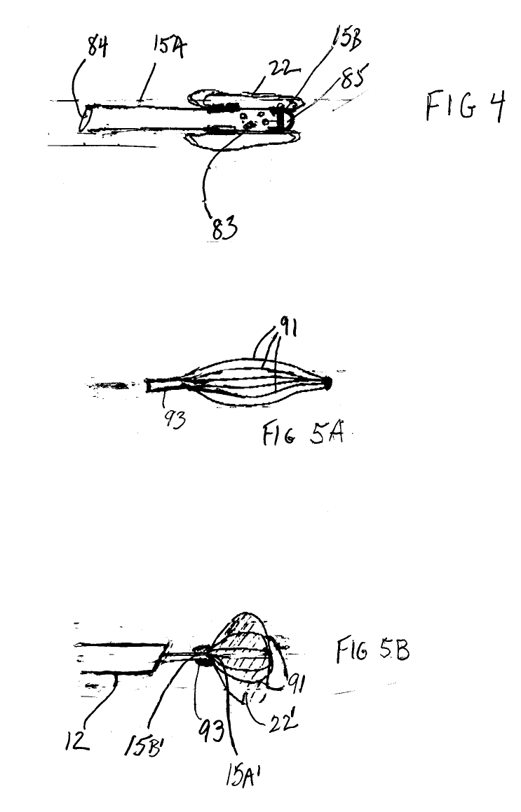

Turning now to FIGS. 2 and 3, the surgical assembly 10 is seen in more detail with the balloon instrument 14 shown in a deflated orientation and with a modified handle 20a for the needle. In FIG. 2, the balloon instrument 14 is shown with the balloon 22 located beyond the distal tip 18 of the needle such that it may be inflated (as seen in FIG. 1) without concern that it will be punctured by the needle tip 18. In FIG. 3, the balloon instrument 14 is shown with the balloon 22 deflated and withdrawn inside the needle 12. As seen best in FIG. 2, and as described hereinafter with reference to FIG. 4, the distal end of the hollow needle shaft 15 defines a plurality of side or peripheral holes 83 through which a fluid such as air or water which is injected through the hollow 84 of the hollow shaft 15 of the balloon instrument 12 will flow in order to inflate the balloon 22.

The distal end of the balloon instrument 14 is seen in FIG. 4. In FIG. 4, the distal end 15A of the hollow needle shaft 15 defines peripheral holes 83. A toroidal balloon or bladder 22 typically formed from polyurethane, latex, other materials well known in the balloon catheter arts is affixed to the distal end 15A by gluing, sonic welding, or affixing in other manners well known in the balloon catheter arts. The balloon is designed to open to a desired size; e.g., 2.5 cm diameter. The distal tip 15B of the shaft is closed off preferably using a plug 85 to prevent the balloon inflation fluid from exiting the shaft 15. The plug 85 may be friction fit to the inside surface of the hollow shaft 15, or may be otherwise affixed thereto. The plug 85 is preferably relatively soft and may be formed from silicone or any other desirable material which can provide a non-traumatic presenting surface for the balloon instrument 14.

The distal end of a second embodiment of a balloon instrument is seen in FIGS. 5A and 5B. In the second embodiment the distal tip 15B′ of the hollow balloon instrument shaft 15′ is open and the distal end 15A′ of the hollow balloon instrument shaft does not include side or peripherals holes. Attached to the distal tip 15B′ is a balloon or bladder structure including a plurality of thin, flexible, resilient support wires 91 and a bladder or balloon 22′ preferably surrounding the wires and attached to the wires in a manner well known in the balloon catheter arts. The balloon 22′ is typically formed from polyurethane, or latex, or other materials well known in the balloon catheter arts. The proximal end of the wires preferably terminate in a collar 93 which fits over or inside of the hollow distal tip 15B′ of the shaft 15′ and is attached by friction, gluing, sonic welding, or in other manners well known in the balloon catheter arts. When the balloon 22′ is not inflated, the wires assume a generally straight configuration as seen in FIG. 5A. When the balloon 22′ is inflated, the balloon 22′ and hence the wires 91 assume a spherical configuration as seen in FIG. 5B. Upon deflation of the balloon 22′, the wires 91 and balloon 22′ reassume the configuration seen in FIG. 5B due to the resilience of the wires.

The surgical assemblies of the invention may be used during laparoscopic surgery instead of using extra trocars and laparoscopic instruments. The balloon instrument 14 is particularly suitable for use as a soft tissue dissector, an expander, a retractor, or an organ manipulator. In particular, with the balloon instrument 14 partially inserted in the needle 12 (as seen in FIG. 3) and optionally locked relative to each other by the first fixing element 50, the needle 12 is used to puncture the skin and advance into the body (e.g., the abdomen). At a desired location (typically under guidance of an already inserted scope), the movement of the needle is stopped. The balloon instrument 14 is then unlocked (if previously locked) and preferably advanced until the proximal end of the balloon 22 (or 22′) is located beyond the sharp end 18 of the needle. The needle and balloon instrument may then be further advanced until the balloon is located at a desired location in the body, or the needle may be kept stationary and the balloon instrument advanced alone. After the balloon 22 (22′) is located beyond the sharp end 18 of the needle, the balloon may be inflated by injection of a fluid such as water or air into and through the hollow shaft 15 of the balloon instrument 14 and out into the balloon. A valve may be closed to prevent deflation of the balloon. Prior to inflation, or after inflation, the first fixing element or system 50 may be used to fix the needle relative to the balloon instrument 14 to prevent inadvertent puncture of the balloon (during or after inflation). If the balloon instrument 14 is to be used as a tissue dissector, the balloon instrument 14 is inserted into the tissue layer prior to inflation of the balloon 22 (22′). Similarly, if the balloon instrument is to be used as a retractor or manipulating instrument, the balloon 22 (22′) will typically be inflated first so that the assembly of the needle with the balloon instrument fixed relative thereto may be manipulated relative to the body wall (e.g., to lift, push, or otherwise move a structure such as an organ). Regardless, when the needle and balloon are in a desired location in the body, the second fixing element 80 can be slid along the needle and into engagement with the skin of the patient, thereby fixing the balloon 22 (22′) at a desired location in the body. At any time, the balloon can be deflated, e.g., by opening the valve. The surgical assembly 10 can be pulled out of the body (preferably with the balloon instrument 14 first moved backward at least partially relative to the needle 12) leaving just a small puncture mark which will often heal without a scar.

It is noted that because of the small diameter of the surgical assembly, withdrawal of the needle assembly from the abdomen will not cause desufflation of the abdomen, and should not require stitching to close the wound. It is also noted that because of the small diameter of the surgical assembly the elimination of a trocar port, the surgical assembly can be easily moved in any direction (i.e., it can be easily angled) during surgery.

The surgical assembly of the invention thereby accomplishes the objects of the invention with a minimum number of parts and may be used to replace expensive trocar assemblies and laparoscopic instruments.

There have been described and illustrated herein several embodiments of a minimally invasive surgical assembly and methods for the use thereof. While particular embodiments of the invention have been described, it is not intended that the invention be limited thereto, as it is intended that the invention be as broad in scope as the art will allow and that the specification be read likewise. Thus, while particular materials for making the needle and balloon instrument have been disclosed, it will be appreciated that other materials may be used as well, and while exemplary diameters for the balloon and lengths for the needle and flexible tube have been disclosed, other balloon diameters and needle and tube lengths can be utilized. In addition, while particular fixing elements and systems have been disclosed for fixing the balloon instrument relative to the needle, it will be understood that other mechanisms can be used. Further, while the balloon instrument and needle have been shown as being straight, because of their small diameter they may be bent together by the user, or one or both may be formed with a bend (arc). Moreover, while particular configurations have been disclosed in reference to the handles of the surgical instrument and the needle have been disclosed, it will be appreciated that other configurations could be used as well. In addition, while the needle was described as being a particular size and having a sharp end with a certain angle, it will be appreciated that other size needles can be used and the sharp can be at different angles. It will therefore be appreciated by those skilled in the art that yet other modifications could be made to the provided invention without deviating from its spirit and scope as claimed.

What is claimed is:

1. A surgical assembly, comprising:
a) a hollow needle having an outer diameter of at most 3.0 mm and a sharp distal end; and
b) a surgical instrument having a hollow shaft which extends through said hollow needle and an inflatable member coupled to and located at a distal end of said hollow shaft, said surgical instrument being movable relative to said hollow needle.

2. A surgical assembly according to claim 1, wherein:
said hollow needle has an inner surface and said hollow shaft has an outer surface, and said outer surface and said inner surface are sized so that at least a portion of said shaft interferingly slides against said inner surface of said needle, thereby forming a seal.

3. A surgical assembly according to claim 1, further comprising:
a first fixing means coupled to said surgical instrument and said needle for fixing a relative location of said surgical instrument and said needle.

4. A surgical assembly according to claim 3, further comprising:
second fixing means coupled to and movable relative to said needle for fixing a relative location of said needle to a patient.

5. A surgical assembly according to claim 1, wherein:
said needle has a sharpened end angled at substantially 35°.

6. A surgical assembly according to claim 1, wherein:
said inflatable member comprises a balloon.

7. A surgical assembly according to claim 6, wherein:
said hollow shaft has a distal end defining side openings, and said balloon is a toroidal balloon.

8. A surgical assembly according to claim 7, wherein:
said surgical instrument further comprises a plug received in the distal end of said hollow shaft.

9. A surgical assembly according to claim 6, wherein:
said inflatable member further comprises wires coupled to said balloon.

10. A surgical assembly according to claim 9, wherein:
said inflatable member includes a proximal collar coupled to a distal end of said hollow shaft.

11. A surgical assembly according to claim 1, wherein:
said surgical instrument includes means for coupling said hollow shaft to a fluid source.

12. A surgical assembly according to claim 1, wherein:
said surgical instrument consists essentially of said hollow shaft, said inflatable member, a handle or knob for moving said shaft together with said inflatable member, and means for coupling said hollow shaft to a fluid source.

13. A surgical assembly according to claim 1, wherein:
said means for coupling includes a valve.

14. A surgical assembly according to claim 12, wherein:
said needle consists essentially of a hollow shaft having said outer diameter of substantially 3 mm or smaller, said sharp distal end, and a proximal handle or knob.

15. A surgical assembly according to claim 14, wherein:
said surgical assembly consists essentially of said hollow needle, said surgical instrument, and means for fixing said surgical instrument relative to said needle.

16. A surgical assembly according to claim 14, wherein:
said surgical assembly consists essentially of said hollow needle, said surgical instrument, means for fixing said surgical instrument relative to said needle, and means for fixing said hollow needle relative to a body cavity into which said sharp distal end is placed.

17. A surgical method, comprising:
a) obtaining a surgical assembly having a hollow needle having an outer diameter of at most 3.0 mm and a sharp distal end and a surgical instrument having a hollow shaft which extends through said hollow needle and an inflatable member coupled to and located at a distal end of said hollow shaft, said surgical instrument being movable relative to said hollow needle;
b) with said inflatable member located inside said hollow needle, using said sharp distal end of said hollow needle to insert a distal portion of said surgical assembly into a cavity of a patient;
c) moving said surgical instrument forward relative to said needle such that a proximal end of said inflatable member is located distal of said sharp distal end;
d) inflating said inflatable member with fluid;
e) manipulating an object in the cavity using said inflatable member;
f) deflating said inflatable member; and
g) withdrawing said surgical assembly from the cavity.

18. A method according to claim 17, further comprising:
locking said needle and said surgical instrument together prior to said manipulating.

19. A method according to claim 18, wherein
said manipulating comprises moving said needle and said surgical instrument together.

20. A method according to claim 17, wherein:
said manipulating comprises dissecting tissue in said cavity by said inflating.
